(19) United States
(12) Patent Application Publication
(10) Pub. No.: US 2025/0091966 A1
(43) Pub. Date: Mar. 20, 2025

(54) Silicate Solubilizing Streptomyces Sp. CS13-6 and Uses Thereof

(71) Applicant: INSTITUTE OF BIOLOGY, HEBEI ACADEMY OF SCIENCE, Hebei (CN)

(72) Inventors: Huicai CHENG, Hebei (CN); Yanhua XI, Hebei (CN); Ling YANG, Hebei (CN); Qiang HE, Hebei (CN)

(21) Appl. No.: 18/558,311

(22) PCT Filed: Mar. 23, 2023
(86) PCT No.: PCT/CN2023/083423

§ 371 Date: Oct. 31, 2023

(30) Foreign Application Priority Data
Sep. 8, 2022 (CN) 202211094490.7

(51) Int. Cl. C05F 11/08; C05G 3/80; C12N 1/20; C12R 1/465

(52) U.S. Cl. CPC C05F 11/08; C05G 3/80; C12N 1/205; C12R 2001/465

(57) ABSTRACT

The present invention relates to an efficient silicate solubilizing Streptomyces sp. CS13-6 with a preservation No. of CGMCC No. 25523, which has high capabilities of silicate solubilization, dissolving phosphorus and potassium, fixing nitrogen, and producing siderophore and IAA. The strain can grow well at pH 5-10 and 20-40° C. In addition, it can grow normally when the NaCl content is 4%, and has high acid, alkali and salt resistance and wide temperature adaptability. The present invention further discloses use of the strain in promoting plant growth, improving soil quality and the like. The strain can be used as an active ingredient for preparation of biological silicon fertilizers, soil conditioners, etc., so as to effectively improve the nutrient structure in the soil, improve soil quality, reduce the use of chemical fertilizers, and achieve a purpose of improving quality and increasing yield.

### TECHNICAL FIELD

The present invention relates to the technical field of environmental microorganisms, particularly to silicate solubilizing Streptomyces sp. CS13-6 and uses thereof.

### BACKGROUND ART

In China, more than 50-80% of farmland soils seriously lacks silicon (Si), and more than 20% of soils is polluted by heavy metals such as Cd, As and Pb, etc., and the diversity of microorganisms in soils is out of balance. Consequently, the agricultural production and the quality and safety of the agricultural products are seriously affected. The application of silicon fertilizers can not only improve quality and yield, but also adjust the pH of the soil, improve the soil structure and remediate polluted soil, and can obviously promote the level of soil productivity. More and more attention has been paid to silicon fertilizers, and a large number of small and medium-sized silicon fertilizer enterprises have emerged at the right moment in China. According to incomplete statistics, the annual production capacity of silicon fertilizers in China is more than one million tons per year, but there are still a huge gap and a development space for silicon fertilizer production for Chinese silicon fertilizer market with an annual demand of 30-50 million tons.

At present, there are two main types of silicon fertilizers produced and applied in China: one type of silicon fertilizers are synthetic fertilizers, such as dicalcium silicate (2CaO·SiO₂), monocalcium silicate (CaO·SiO₂), calcium magnesium silicate (CaO·MgO·SiO₂), sodium metasilicate, etc.; silicon fertilizers produced through a synthetic process has 50% or higher effective silicon content, but they are expensive and difficult to popularize, and may destroy the soil structure and reduce the organic matter content during long-term application. The other type of silicon fertilizer involves silicon fertilizers made from various industrial solid wastes such as water-quenched blast furnace slag, phosphorus slag, fly ash and waste glass, etc. Although China has abundant raw materials for silicon fertilizer production and the technical level is constantly improved, many industrial solid wastes are restricted seriously when used for producing agricultural fertilizers, owing to the increasingly stringent environmental protection requirements.

Therefore, it is of great significance for the development of green ecological agriculture to screen and seek for new microorganisms with silicate solubilizing function and develop microorganisms that have good effect, low cost and no pollution to the environment, in order to develop the silicon resources in soil.

### CONTENTS OF THE INVENTION

The object of the present invention is to provide a silicate solubilizing Streptomyces sp. CS13-6 that can not only solubilize silicate, but also dissolve phosphorus and potassium, fix nitrogen and produce siderophore and IAA, and uses thereof.

The present invention employs the following technical solution:
a Streptomyces sp. CS13-6, preserved on Aug. 12, 2022 in the China General Microbiological Culture Collection Center (CGMCC) (address: Institute of Microbiology, Chinese Academy of Sciences, Building No. 3, Courtyard No. 1, Beichen West Road, Chaoyang District, Beijing), and the preservation No. is CGMCC No. 25523.

Furthermore, the silicate solubilizing Streptomyces sp. CS13-6 has a silicate solubilizing capability.

Furthermore, the silicate solubilizing Streptomyces sp. CS13-6 has the capabilities of dissolving phosphorus and potassium and fixing nitrogen.

Furthermore, the silicate solubilizing Streptomyces sp. CS13-6 has a siderophore and IAA production capability.

Furthermore, the silicate solubilizing Streptomyces sp. CS13-6 can grow well in an initial culture medium with a pH value of 5-10.

Furthermore, the silicate solubilizing Streptomyces sp. CS13-6 has the capabilities of silicate solubilization, dissolving phosphorus and potassium, fixing nitrogen, and resisting acid and alkali.

Use of the above-mentioned silicate solubilizing Streptomyces sp. CS13-6 in plant growth promotion and/or soil remediation.

A bio-silicon fertilizer and/or a soil conditioner are produced by using the above-mentioned silicate solubilizing Streptomyces sp. CS13-6 as an active ingredient.

The present invention attains the following beneficial effects:

The silicate solubilizing Streptomyces sp. CS13-6 in the present invention has a significant advantage in acidic or alkaline soil resistance and has a strong colonization capability, and can be used for improving acidic and alkaline soil and improving quality; in addition, it can provide a high-quality strain resource for the development of functional microorganisms that can solubilize silicate and dissolve phosphate and potassium, and has practical economic benefits and broad application prospects.

The silicate solubilizing Streptomyces sp. CS13-6 of the present invention can be used for ecological soil reclamation, silicon-deficient soil improvement and the like in tailing areas.

When the silicate solubilizing Streptomyces sp. CS13-6 of the present invention is applied to soil, it can not only solubilize silicate, but also dissolve phosphorus and potassium, fix nitrogen and produce siderophore and IAA, thereby not only improves soil nutrients, but also significantly promotes plant growth.

### DESCRIPTION OF DRAWINGS

FIG. 1 is a colony diagram of Streptomyces sp. CS13-6;

FIG. 2 shows aerial mycelia of Streptomyces sp. CS13-6;

FIG. 3 shows spores of Streptomyces sp. CS13-6.

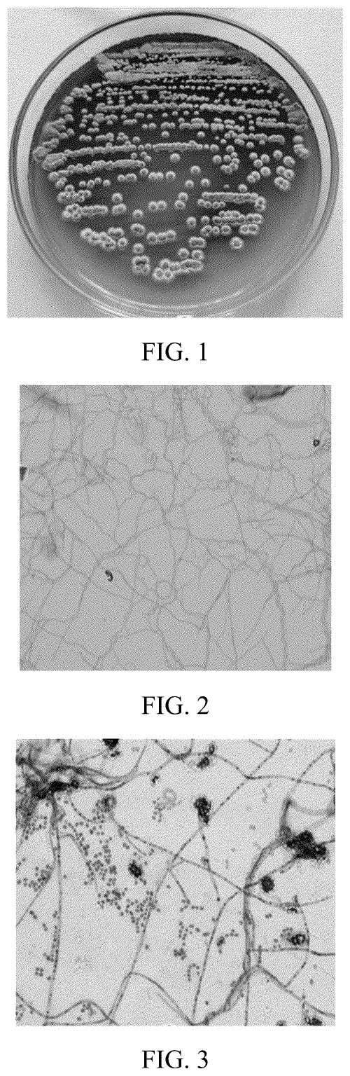

### EMBODIMENTS

The present invention will be further described below in examples, with reference to the accompanying drawings. The scope of protection of the present invention is not limited to those examples, and any modification made by those skilled in the art within the scope defined by the claims also falls in the scope of protection of the present invention.

### Example 1

### Isolation and Preservation of Streptomyces sp. CS13-6

Streptomyces sp. CS13-6 was isolated from the tailing sand samples of iron mine in Chengde, Hebei Province, China through primary screening with a gradient dilution plate method and secondary screening with bioleaching.

The specific process is as follows: the collected sample was ground and screened through a 200-mesh screen to prepare three bacterial suspensions with dilutions of 10⁻³, 10⁻⁴ and 10⁻⁵, each of the bacterial suspensions was evenly spread on an Alexandrov culture medium, and cultured in a constant-temperature incubator at 30° C. for 2-3 days; then, individual colonies in different forms were selected and streaked repeatedly for 2-3 times till pure cultures were obtained; the strain was preliminarily judged as actinomycetes according to the colony morphology, and was transferred to the slant surface of No. 1 Gauze's Medium, then the culture medium was preserved in a refrigerator at 4° C. for later use.

Iron tailing sands were used as the substrate, and the individual colonies obtained from the primary screening were inoculated into a leaching and desiliconizing medium for secondary screening of silicate solubilizing capability, based on a criterion that the effective silicon content in the fermentation broth increases. A sample was taken on the fifth day of fermentation, and the supernatant was obtained by centrifugation of the fermentation broth.

The effective silicon content was determined by silicon-molybdenum blue colorimetry.

### Culture Medium in Use:

Alexandrov culture medium: 5.0 g sucrose, 2.0 g Na₂HPO₄, 0.5 g MgSO₄·7H₂O, 0.5 g FeCl₃, 0.1 g CaCO₃, 1.0 g soil mineral sample, 15-20 g agar, 1 L H₂O, and pH=7.0-7.4.

Leaching and desiliconizing medium: 10 g glucose, 0.2 g KH₂PO₄, 0.2 g MgSO₄·7H₂O, 0.2 25 g NaCl, 0.2 g CaCl₂·2H₂O, 5 g CaCO₃, 1 L H₂O, and pH=7.0-7.2.

No. 1 Gauze's Medium: 1 g KNO₃, 20 g soluble starch, 0.5 g NaCl, 0.5 g K₂HPO₄·3H₂O, 0.5 g MgSO₄·7H₂O, 0.01 g FeSO₄·7H₂O, 15-20 g agar (for solid medium), 1 L H₂O, and pH=7.4-7.6.

The strain CS13-6 grew well on the No. 1 Gauze's Medium, the colony was white in the initial stage, and the edge of the colony was neat with protrusions, the aerial mycelia were white and branched, a blue soluble pigment was produced in the later stage. The spore morphology had an oval shape and the spore chains were spiral. The strain could hydrolyze starch and liquefy gelatin, and could grow on cellulose, but couldn't produce hydrogen sulfide. The strain could utilize a variety of carbon sources (glucose, mannitol, D-galactose, L-rhamnose, D-fructose, sucrose and maltose) and nitrogen sources (ammonium sulfate, sodium nitrate, peptone and yeast extract powder).

The mycelia which had been cultured for 72 h were sent to a sequencing company for whole genome sequencing by means of MGI2000 second-generation and ONT third-generation sequencing, and the assembled genome was subjected to taxonomic identification. It was confirmed that the strain was the closest to Streptomyces regalis in taxonomy, and the average nucleotide identity (ANI) between the two genomes was 92.43 (ANI is defined as the average base similarity between the homologous fragments of two microbial genomes, and is characterized in that it achieves high discrimination among closely related species; the genomic ANI of the same species is higher than 95%, while the genomic ANI of different species is lower than 95%; therefore, 95% ANI is often used as a criterion for species division and species clustering). The ANI value was lower than 95%, indicating that the strain was a new species in Streptomyces.

The strain CS13-6 was preserved on Aug. 12, 2022 in the China General Microbiological Culture Collection Center (CGMCC) (address: Institute of Microbiology, Chinese Academy of Sciences, Building No. 3, Courtyard No. 1, Beichen West Road, Chaoyang District, Beijing), its taxonomic name is Streptomyces sp., and its preservation No. is CGMCC No. 25523.

### Example 2

### Silicate Solubilizing Feature of Streptomyces sp. CS13-6

A small amount of pure strain CS13-6 was taken with an inoculating loop and inoculated into a 250 mL triangular flask containing 50 mL No. 1 Gauze's Medium (as in the Example 1), and cultured at 28° C. for 72 h while shaking at 180 rpm. to obtain an activated strain.

The CS13-6 bacterial solution was inoculated in leaching and desiliconizing mediums respectively at a volume ratio of 5% (as in the Example 1). The influences of leaching temperature, initial pH and rotation speed on the desiliconizing performance of the strain CS13-6 were investigated respectively. The fermentation broth on the fifth day was taken and centrifuged at 8,000 rpm for 10 min, and the supernatant was filtered through a 0.22 μm filter membrane. The effective silicon content in the cultured fermentation broth was determined by means of silicon-molybdenum blue colorimetry according to NY/T 1121.15-2006.

The experimental result indicated that the optimal conditions for desilication of strain CS13-6 were as follows: temperature: 28-30° C., initial pH: 6.0-7.0, rotation speed: 150-180 rpm.; the effective silicon content reached 43.2 mg/L.

### Example 3

### Determination of Phosphorus and Potassium Dissolution and Nitrogen Fixation Capabilities of Streptomyces sp. CS13-6

A seed plate of the strain CS13-6 was prepared and then it was inoculated in a NBRIP detection medium for phosphorus dissolution and a potassium-dissolving medium. After the strain was cultured in a constant temperature incubator at 30° C. for three days, the growth condition of the strain was observed and whether there was a dissolution circle was checked.

The calculation method is: dissolution capability=diameter D of dissolution circle/diameter d of colony.

NBRIP medium: 10 g Glucose, 5 g Ca₃(PO₄)₂, 0.5 g (NH₄)₂SO₄, 0.25 g MgSO₄·7H₂O, 0.2 g KCl, 5 g MgCl₂·6H₂O, 15 g agar, 6 mL 0.4% bromophenol blue, 1 L H₂O, and pH=7.0-7.2.

Potassium-dissolving medium: 10 g sucrose, 1 g Na₂HPO₄, 0.5 g (NH₄)₂SO₄, 1 g MgSO₄·7H₂O, 0.2 g yeast powder, 0.1 g NaCl, 0.1 g CaCO₃, 0.005 g FeCl₃, 5 g potash feldspar, and 1 L H₂O.

The testing result indicated: in the NBRIP detection mediums for phosphorus dissolution and potassium-dissolving medium, there were obvious transparent circles around the CS13-6 colony, which indicated that CS13-6 had certain phosphorus and potassium dissolving capabilities.

A seed plate of the strain CS13-6 was prepared, and some colonies were picked and inoculated in a nitrogen-fixing activity detection medium, and cultured at 30° C. for 3 days, and the growth condition of the strain was observed. After three transfers, the strain could still grow, which indicated that CS13-6 had an inherent nitrogen fixation capability.

Nitrogen-fixing activity detection medium (Ashby nitrogen-free medium): 10 g mannitol, 0.2 g KH₂PO₄, 0.2 g MgSO₄·7H₂O, 0.2 g NaCl, 0.2 g CaSO₄·2H₂O, 5 g CaCO₃, 1 L H₂O, and pH=7.2.

The testing result indicated: the strain CS13-6 could still grow in the nitrogen-fixing medium after three consecutive transfers, which indicated that the strain had a nitrogen fixation capability.

### Example 4

### Determination of Siderophore and IAA Production Capability of Streptomyces sp. CS13-6

Siderophore production capability: A seed plate of the strain CS13-6 was prepared, and some colonies were picked by means of an inoculating needle and inoculated in a CAS detection medium, and cultured at 30° C. for 3 days. If a yellow-green halo appeared, it indicated that siderophore was produced.

IAA secretion capability: a seed plate of the strain CS13-6 was prepared, some colonies were inoculated into No. 1 Gauze's Medium (containing 100 mg/L L-tryptophan), and the medium was placed on a shaking table (30° C., 180 rpm) and cultured for 3-5 days; then, after centrifugation at 8,000 rpm, 50 μL supernatant was taken, 50 μL Salkowski colorimetric solution was added into the supernatant, and the solution was dropped on a white porcelain plate and developed in a dark environment for 30 min. Appearance of pink indicates that the result is positive and the strain can secrete IAA; the darker the color is, the higher the secretion intensity is; if there is no color change, it indicates that the result is negative and the strain can't secrete IAA.

The testing result indicated that the strain CS13-6 has the capability of producing IAA and siderophore, and has a strong capability of promoting plant growth.

### Example 5

### Stress Resistance Experiment of Streptomyces sp. CS13-6

A seed plate of the strain CS13-6 was prepared, and some colonies were picked for streak inoculation in plates of No. 1 Gauze's Medium with different initial pH values (2, 3, 4, 5, 6, 7, 8, 9, 10, 11, and 12) and NaCl contents (0, 0.5%, 2%, 4%, 6%, 8%, 10%, 12%, and 15%), and inoculated at 30° C. constant temperature for 2-3 days. At the same time, the colonies of the strain CS13-6 were streaked and inoculated to plates of No. 1 Gauze's Medium, and cultured at different temperatures (4° C., 10° C., 20° C., 30° C., 40° C., 45° C. and 50° C.) for 2-3 days, and the growth condition of the colonies was observed.

The result indicated that the strain CS13-6 could grow normally at pH 5-10. The strain could still grow normally when the NaCl content in the culture medium is 4%, and the optimal NaCl concentration was 0-0.5%, which indicated that the strain had certain salt resistance. The strain CS13-6 could grow well at 20-40° C., which indicated that the strain CS13-6 had strong tolerance to adverse conditions, and has high acid and alkali resistance and wide temperature adaptability.

### Example 6

### Effect of Streptomyces sp. CS13-6 on Vegetable Growth

The Streptomyces sp. CS13-6 was inoculated on No. 1 Gauze's Medium, cultured in a constant temperature incubator at 28° C. for 5-8 days; some spores on the surface of the culture medium were scraped and placed in 20 mL sterile water containing glass beads, and a bacterial suspension was prepared at 28° C. by shaking at 220 rpm for 20-30 minutes; a blood counting chamber was used for counting, and the bacterial suspension was diluted to four different concentrations according to a gradient dilution method; the spore concentrations of the Streptomyces sp. CS13-6 were 6×10⁵ cfu·g⁻¹, 6×10⁶ cfu·g⁻¹, 6×10⁷ cfu·g⁻¹ and 6×10⁸ cfu·g⁻¹ respectively.

Healthy lettuce seeds with uniform size and full kernel were selected randomly, and the surfaces of the seeds were disinfected with 70% ethanol and 3% NaClO aqueous solution. Lettuce seedling: the substrate was composed of a part of garden soil, iron tailing sands and vermiculite, and was evenly spread on a seedling tray; lettuce seeds were evenly spread on the seedling tray, and covered with the substrate having a thickness of about 0.5 cm to ensure that the seeds were not exposed after watering. After completion of the seeding, the seedling tray was watered thoroughly and cultivated in a greenhouse.

Lettuce planting: when the lettuce grew to a stage with three leaves and one heart, the lettuce seedlings with uniform size were selected from the seedling tray and transplanted into a flowerpot with a diameter of 15 cm, and Streptomyces sp. CS13-6 at different concentrations were added into the potted soil. Five treatments were set in the experiment: clean water control was used as a blank control CK, and the other four treatments were Streptomyces sp. CS13-6 with spores concentrations of 6×10⁵ cfu·g⁻¹, 6×10⁶ cfu·g⁻¹, 6×10⁷ cfu·g⁻¹ and 6×10⁸ cfu·g⁻¹ respectively; 10 pots were used for each treatment, three repetitions were performed; the room temperature was set to 28° C. and the light duration was 12 hours during daytime; the room temperature was set to 24° C. during night time and the dark duration was 12 hours; the single plant yield, plant height, number of leaves, leaf length and leaf width of the lettuce plants were measured after 30-40 days.

The effect of Streptomyces sp. CS13-6 on the growth of potted lettuce is shown in Table 1. The Streptomyces sp. CS13-6 with different spore concentrations can all promote the growth of lettuce. With the increase of the quantity of spores, all growth indexes of lettuce are obviously improved. When the quantity of spores of Streptomyces sp. CS13-6 is 6×10⁷ cfu·g⁻¹, the single plant yield of lettuce is 57.3 g, the plant height is 18.6 cm and the number of leaves is 9.8; the leaf length and width are 17.9 cm and 13.1 cm respectively, which are significantly higher than those of the control group CK, indicating that Streptomyces sp. CS13-6 has an obvious promotion effect on the growth of potted lettuce, and the most significant concentration is 6×10⁷ cfu·g⁻¹.

TABLE 1
Effect of Streptomyces Sp. CS13-6 on the Growth of Lettuce Plants

| | Plant | Single plant | Number of | Leaf | Leaf |
|---|---|---|---|---|---|
| Treatment | height/ | yield, | leaves | length | width |
| (CFU · g⁻¹) | cm | g/plant | (piece) | (cm) | (cm) |
| Control group | 16.2 c | 42.5 d | 8.9 c | 16.4 c | 10.9 d |
| 6 × 10⁵ | 17.0 b | 48.5 c | 9.4 b | 16.9 b | 11.8 c |
| 6 × 10⁶ | 17.4 b | 52.3 b | 9.6 b | 17.0 b | 12.5 b |
| 6 × 10⁷ | 18.6 a | 57.3 a | 9.8 a | 17.9 a | 13.1 a |
| 6 × 10⁸ | 17.5 b | 56.8 a | 9.5 b | 17.8 a | 12.8 b |

Note:
the result is the average value (standard error) of three repeated measurements; different lowercase letters indicate significant differences between treatments (P < 0.05).

### Example 7

### Effect of Streptomyces sp. CS13-6 on Soil and Plant Growth

The strain CS13-6 was fermented and cultured in No. 1 Gauze's liquid Medium at 28° C. while shaking at 200 rpm for 3-5 days to prepare a bacterial suspension (or prepare a spore suspension and adjust the spore concentration to 1×10⁸ CFU/mL) for later use, and a substrate composed of a part of garden soil, iron tailing sands and vermiculite was added at a ratio of 100 mL·kg⁻¹. As for the control group, the same volume of sterile water was mixed into the substrate to carry out potted corn experiment. The pots are placed in a greenhouse at 25° C.; after germination of the seeds, three best-growing seedlings were kept in each pot, 10 replicates were used for each group, and water was replenished regularly. In addition, for the experimental group, 5-10 mL fermentation broth or spore suspension was replenished into each pot every 10-15 days; for the control group, the same amount of sterile water was added; the morphological characteristics of the plants were observed and recorded regularly. After 2 months, the soil and the above ground indexes and root growth conditions of the plants (corn) were measured.

TABLE 2
Effect of Streptomyces Sp. CS13-6 on the Growth of Corn Plants

| | | | Root-top | Chlorophyll |
|---|---|---|---|---|
| | Plant | Dry weight | ratio | content |
| | height/ | of plant | (g/g dry | (mg/g fresh |
| | cm | (g/plant) | weight) | weight) |
| Control group | 27.02 | 1.36 | 0.61 | 6.35 |
| CS13-6 | 35.07 | 2.21 | 1.35 | 16.51 |

TABLE 3
Effect of Streptomyces Sp. CS13-6 on Soil Fertility

| | | Rapidly | Rapidly | |
|---|---|---|---|---|
| | Available | available | available | Total |
| | silicon | potassium | phosphorus | nitrogen |
| | increase | increase | increase | increase |
| | rate | rate | rate | rate |
| Control group | — | — | — | |
| CS13-6 | 93.2% | 47.1% | 62.5% | 35.4% |

The result indicates (see Table 2): the corn plants in the experimental group are significantly better than those in the control group in terms of plant growth and root system, especially in terms of plant height, dry weight and chlorophyll content. The analytical result is shown in Table 3. Besides, the contents of available silicon, rapidly available potassium and rapidly available phosphorus in soil can be significantly increased.

The present invention is described in detail according to the above examples. It should be noted that the above examples are only for illustrating the present invention. Those skilled in the art may design various alternatives and improvements to the present invention without departing from the spirit and essence of the present invention, but all such alternatives and improvements should be understood as falling within the scope of protection of the present invention.

1. A silicate solubilizing Streptomyces sp. CS13-6, wherein its preservation number is CGMCC No. 25523.

2. The silicate solubilizing Streptomyces sp. CS13-6 according to claim 1, wherein it has a silicate solubilizing capability.

3. The silicate solubilizing Streptomyces sp. CS13-6 according to claim 1, wherein it has capabilities of dissolving phosphorus and potassium and fixing nitrogen.

4. The silicate solubilizing Streptomyces sp. CS13-6 according to claim 1, wherein it has siderophore and IAA production capability.

5. The silicate solubilizing Streptomyces sp. CS13-6 according to claim 1, wherein, it can grow well in an initial culture medium with pH 5-10, and has high acid and alkali resistance.

6. The silicate solubilizing Streptomyces sp. CS13-6 according to claim 1, wherein, it can grow well at 20-40° C. and has a wide temperature adaptability.

7. The silicate solubilizing Streptomyces sp. CS13-6 according to claim 1, wherein, it can still grow normally when the NaCl content is 4%, and has salt resistance.

8. The silicate solubilizing Streptomyces sp. CS13-6 according to claim 1, wherein it has capabilities of silicate solubilization, dissolving phosphorus and potassium, fixing nitrogen and resisting acid and alkali.

9. A method for promoting plant growth wherein said method comprises applying to the plant or its surroundings the silicate solubilizing Streptomyces sp. CS13-6 according to claim 1.

10. A bio-silicon fertilizer and/or a soil conditioner produced by using the silicate solubilizing Streptomyces sp. CS13-6 according to claim 1 as an active ingredient.

11. A bio-silicon fertilizer and/or a soil conditioner composition comprising the silicate solubilizing Streptomyces sp. CS13-6 according to claim 1 as an active ingredient.

12. A method for soil remediation wherein said method comprises applying to the soil the silicate solubilizing Streptomyces sp. CS13-6 according to claim 1.
